(19) United States
(12) Patent Application Publication
(10) Pub. No.: US 2017/0326323 A1
(43) Pub. Date: Nov. 16, 2017

(54) KERATINOCYTE STIMULATION FOR WOUND PREVENTION

(71) Applicant: KONINKLIJKE PHILIPS N.V., EINDHOVEN (NL)

(72) Inventor: JONATHAN SAYER GRASHOW, PITTSBURGH, PA (US)

(21) Appl. No.: 15/528,236

(22) PCT Filed: Oct. 29, 2015
(86) PCT No.: PCT/IB2015/058354

§ 371 Date: May 19, 2017

Related U.S. Application Data
(60) Provisional application No. 62/084,012, filed on Nov. 25, 2014.

(51) Int. Cl. A61M 16/06; A61M 16/20; A61M 16/08; A61K 31/00; A61K 9/00; A61K 8/00

(52) U.S. Cl. CPC A61M 16/0666; A61M 16/0816; A61M 16/0683; A61M 16/0633; A61M 16/208; A61K 9/00; A61K 8/00; A61K 31/00; A61M 2205/02; A61M 2205/0238; A61M 2207/00

(57) ABSTRACT

An apparatus structured to contact external tissue, such as skin, of a user includes a portion that has a component, which is effective to stimulate keratinocytes in the skin of the user. The portion is structured to directly or indirectly engage the external tissue. The portion can include a coating applied to a surface of the apparatus or, alternately, the portion can include a piece or part of the apparatus that has the component incorporated in its material composition. In one implementation, the component is an agonist of olfactory receptors which are expressed by skin cells, and the component is effective to stimulate the skin cells for enhanced wound healing.

### CROSS-REFERENCE TO RELATED APPLICATIONS

This patent application claims the priority benefit under 35 U.S.C. §119(e) of U.S. Provisional Application No. 62/084,012, filed on Nov. 25, 2014, the contents of which are herein incorporated by reference.

### BACKGROUND OF THE INVENTION

### 1. Field of the Invention

The present invention pertains to devices that have a surface that is structured to contact the skin or other external tissue (e.g., face, lips, etc.) of the user, such as, without limitation, a respiratory mask used to deliver a breathing gas to a user, and, in particular, to compositions and methods associated with the device that provide keratinocyte stimulation for improved wound prevention for the user.

### 2. Description of the Related Art

A variety of respiratory masks are known that have flexible seals and cover the nose, mouth, or both of a human user. The seals, which are also commonly referred to as cushions, are intended to create a seal against the user's face. Because of the sealing effect that is created, gases can be provided at a positive pressure within the mask for delivery to the airway of the user.

The uses for such masks range from high altitude breathing, i.e., aviation applications, to mining and fire fighting applications, to various medical diagnostic and therapeutic applications. For example, such masks are used in the delivery of continuous positive airway pressure (CPAP) or variable airway pressure, such as a bi-level pressure that varies with the user's respiratory cycle or an auto-titrating pressure that varies with the monitored condition of the user. Typical pressure support therapies are provided to treat a medical disorder, such as sleep apnea syndrome, in particular, obstructive sleep apnea (OSA), or congestive heart failure. During use, such respiratory masks, also often referred to as patient interface devices, are strapped on the head of the patient in order to interface the pressure generating device (e.g., a CPAP machine) with the patient.

A requisite of such respiratory masks is that they provide an effective seal against the user's face to prevent leakage of the gas being supplied, while also providing a comfortable user/seal interface. This problem is most crucial because such masks are typically worn for an extended period of time. One concern in such a situation is that a user may avoid wearing an uncomfortable mask, defeating the purpose of the prescribed pressure support therapy.

Surveys have shown that as much as 70% of the population of users of respiratory masks suffer from facial red marks in some form after using a respiratory mask during therapy. The root causes of red mark formation are the prolonged pressure and shear loading of the skin by the mask cushion in combination with the build-up of moisture and heat in the skin due to the prolonged occlusion. The recovery time varies from minutes to hours. However, in extreme cases, longer-lasting skin damage, ulcers and pressure-sores can occur.

Recently, it has been shown that skin cells can detect odors using the same physiological mechanism as found in the nose, i.e., olfactory receptors. Keratinocytes are the major cell type of the epidermal layer of the skin. Olfactory receptors are the smell receptors that are historically associated with the nasal epithelium. One of the olfactory receptors found in the keratinocytes is OR2AT4. Sandalwood has been shown to be an agonist of OR2AT4. That is, sandalwood stimulates an action or a response, such as a biological action or response. Sandalwood is known in the art and includes commercially available products under the trade names Sandalore® and Brahmanol®. It has been demonstrated that when the olfactory receptors, e.g., OR2AT4, in the skin cells, e.g., keratinocytes, detect specific odors, such as sandalwood, wound healing mechanisms of the skin are significantly enhanced, which can result in faster recovery from skin damage, such as, the red mark formations, ulcers and pressure sores resulting from prolonged pressure and shear loading of the skin by a respiratory mask cushion. In particular, it has been demonstrated that sandalwood activates a cellular signaling cascade that triggers the proliferation and migration of keratinocytes, which accelerates re-epithelialization.

### SUMMARY OF THE INVENTION

Accordingly, it is an object of the present invention to provide an apparatus that has a surface that is structured to contact the skin or other external tissue (e.g., lips) of the user that overcomes the shortcomings of conventional devices by providing improved prevention of wounds or healing of wounds caused by the contact between the surface of the apparatus and the skin or other external tissue of the user.

It is yet another object of the present invention to provide a method of making an apparatus that has a surface that is structured to contact the skin or other external tissue (e.g., lips) that does not suffer from the disadvantages associated with conventional design and manufacturing techniques.

In one embodiment, an apparatus structured to contact external tissue, such as face or lips, of a user during use thereof is provided. The apparatus has a portion that includes a component, which is effective to stimulate keratinocytes in the external tissue.

The portion can be a contacting portion that is structured to directly engage the external tissue of the user. Alternatively, the portion can be a non-contacting portion that is structured to release the component from the portion and to indirectly engage the external tissue of the user.

The portion can be in the form of a coating applied or adhered to a surface of the apparatus. Alternatively, the portion can be a piece or part of the apparatus and the component in the form of an additive in the material composition of the piece or part.

The component can be structured to be an agonist of olfactory receptors, such as, OR2AT4. In one embodiment, the component is sandalwood.

In another embodiment, a method of making an apparatus structured to contact external tissue of a user during use thereof is provided. The method includes obtaining a component, which is effective to stimulate keratinocytes in the external tissue, and forming a portion for the apparatus in a manner wherein the portion includes the component.

In one embodiment, the portion is a coating composition applied or adhered to a surface of the apparatus that directly or indirectly contacts the external tissue of the user. The coating composition can be applied by spraying, brushing, rolling, wiping and adhering to the surface. In another embodiment, the portion is a piece of the apparatus and the component is added to a material composition during manufacture of the piece.

The portion can be structured to at least partially release the component to contact the external tissue of the user.

In still another embodiment, a respiratory mask structured to contact external tissue, such as skin or lips, of a user during use thereof is provided. The mask includes a reservoir structured to provide a component, which is effective to stimulate keratinocytes in the external tissue, into air which flows from an air source connected to the respiratory mask to the user.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1A and 2A are isometric and side elevational views, respectively, of a system adapted to provide a regimen of respiratory therapy to a patient according to an exemplary embodiment of the present invention;

FIGS. 1B and 2B are isometric and side elevational views, respectively, of a system adapted to provide a regimen of respiratory therapy to a patient according to another exemplary embodiment of the present invention;

FIGS. 3 and 4 are schematic diagrams of a cross-section of a component coating as described herein that may be employed in the respiratory mask of the system of FIGS. 1A and 2A; and

FIG. 5 is an isometric view of an alternative nasal cushion that may be used in a respiratory mask wherein the nasal cushion has a component as described herein.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

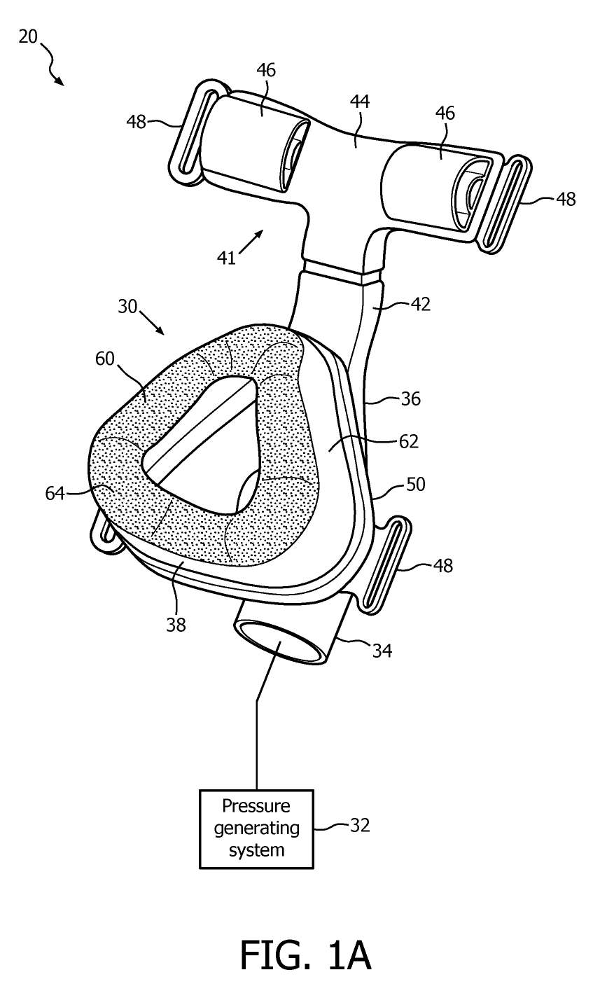

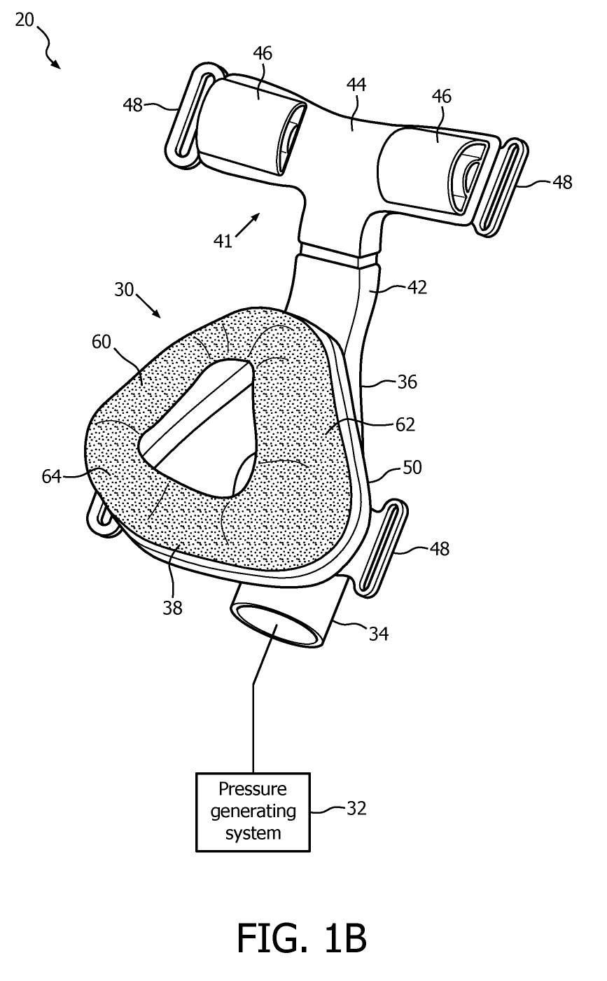

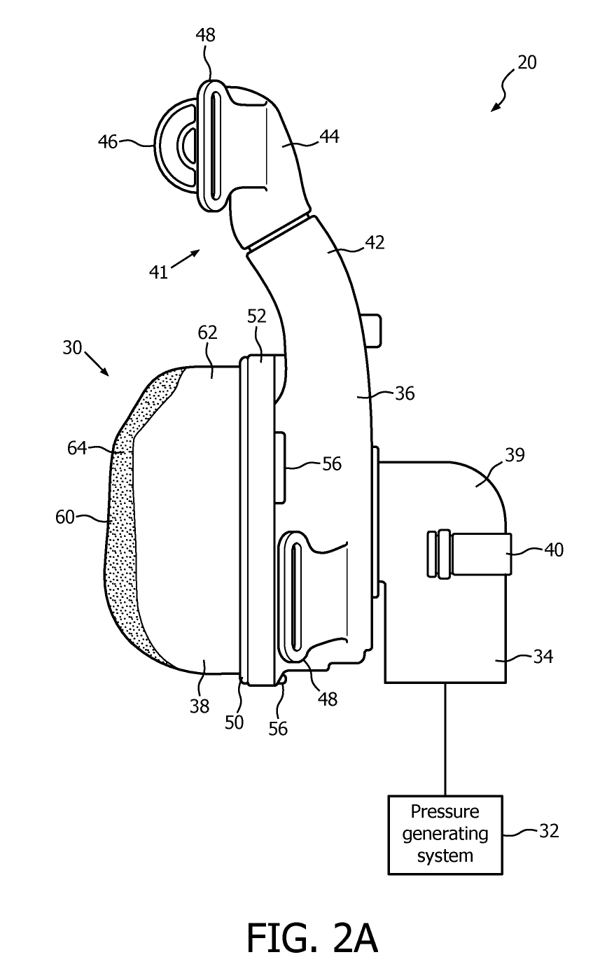

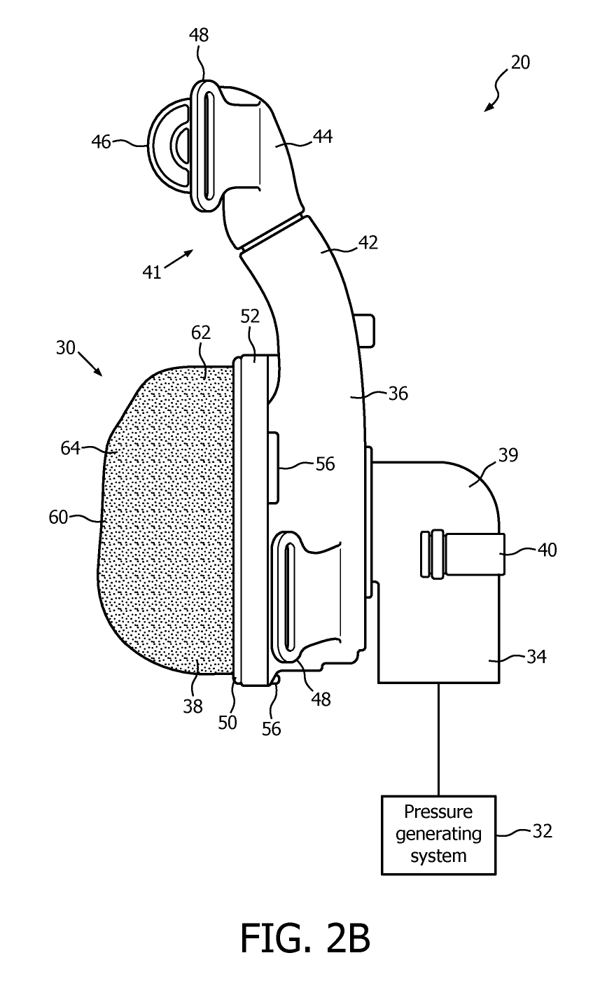

FIGS. 1A and 2A are isometric and side elevational views, respectively, of a system 20 adapted to provide a regimen of respiratory therapy to a patient according to one exemplary embodiment of the present invention. As seen in FIGS. 1A and 2A, system 20 includes a respiratory mask 30 (also referred to as a patient interface device) according to one exemplary embodiment that is shown schematically attached to a pressure generating system 32 via a user circuit 34, as is conventionally known in the art. Pressure generating system 32 is any device capable of generating a flow of breathing gas or providing gas at an elevated pressure. Examples of such pressure generating systems include ventilators, constant pressure support devices (such as a continuous positive airway pressure device, or CPAP device) in which the pressure provided to the user is constant over the user's respiratory cycle, and variable pressure devices (e.g., BiPAP®, Bi-Flex®, or C-Flex™ devices manufactured and distributed by Philips Respironics of Murrysville, Pa.) in which the pressure provided to the user varies with the user's respiratory cycle, and auto-titration pressure support devices.

Respiratory mask 30 includes a shell 36 and a seal 38 attached to shell 36. User circuit 34 is coupled to a port defined in a first side of shell 36 and, in the illustrated embodiment, includes an elbow connector 39 for that purpose. In the exemplary embodiment, user circuit 34 is connected to shell 36 so as to pivot or rotate relative to the shell 36 and may or may not be detachable therefrom. In short, any suitable coupling technique for joining user circuit 34 to shell 36 is contemplated by the present invention.

In the illustrated exemplary embodiment, an exhaust vent assembly 40 is provided in elbow connector 39 for exhausting a flow of gas from mask 30 to ambient atmosphere. Such exhaust vent assemblies are conventionally used in pressure support systems that use a single-limb, i.e., a single conduit, to communicate a flow of gas to an airway of a user. Thus, the present invention contemplates that exhaust vent assembly 40 can be any suitable exhaust vent assembly, and can be located not only on elbow connector 39, but alternatively on mask 30, such as on the shell 36. The particular exhaust vent assembly 40 shown in FIG. 2A corresponds to that described in U.S. Pat. No. 6,851,425, entitled “Exhaust Port Assembly for a Pressure Support System” and owned by the assignee of the present invention.

Respiratory mask 30 can have any one of a number of different configurations, shapes, and sizes. In the illustrated, exemplary embodiment, respiratory mask 30 is a nasal mask structured to cover the nose of the patient wherein shell 36 corresponds to that described in U.S. Pat. No. 7,069,932, entitled “User Interface With Forehead Support System” and owned by the assignee of the present invention. However, other types of respiratory masks, such as, without limitation, a nasal/oral mask, a nasal cushion or a full face mask, which facilitate the delivery of the flow of breathing gas to the airway of a patient, may be substituted for respiratory mask 30 while remaining within the scope of the present invention. Shell 36 is, in the exemplary embodiment, formed from a rigid or semi-rigid material, such as a polycarbonate or an injection molded thermoplastic. In addition, as seen in FIGS. 1A and 2A, respiratory mask 30 includes an adjustable forehead support 41. The forehead support is generally T-shaped and includes a support arm 42 which is slideably connected to a forehead support bracket 44. Forehead support bracket 44 includes a forehead pad 46 disposed on the user contacting side to engage the forehead of the user. It is to be understood that the present invention contemplates that forehead support 41, and its individual components, can have any one of a variety of alternative configurations. The present invention also contemplates that forehead support 41 can be eliminated entirely.

In the illustrated, exemplary embodiment, a headgear (not shown) attaches to respiratory mask 30 via headgear clips 48. Headgear clips 48 attach to straps of the headgear, for example by inserting the straps into slots provided in clips 48. Clips 48 are selectively attachable to shell 36 in any suitable manner. In the illustrated embodiment, clips 48 attach to each side of forehead support bracket 44 and to each side of the lower portion of shell 36. It can thus be appreciated that the headgear and clip 48 can have any configuration so as to be selectively attachable to respiratory mask 30. It is to be further understood that the present invention contemplates eliminating all, or a portion, of clips 48 and attaching the headgear straps to directly the shell 36.

Seal 38, also referred to as a cushion, is, in the exemplary embodiment, a unitary structure made of a soft, flexible, cushiony, elastomeric material, such as, without limitation, silicone, an appropriately soft thermoplastic elastomer (such as thermoplastic polyurethanes (TPU)), latex, polybutadiene, a closed cell foam, or any combination of such materials. In certain embodiments, the seal 38 can be made of a woven or knitted material, e.g., cloth or fabric. The seal 38 includes a first end portion 50 that couples to shell 36. In the illustrated embodiment, first end portion 50 is generally triangular-shaped and attaches to similarly-shaped opening provided in a second side of shell 36. In the illustrated embodiment, shell 36 and first end portion 50 of seal 38 that attaches thereto are both generally planer, i.e., both line in a linear plane. Alternatively, it should be noted that the present invention contemplates that shell 36 and first end portion 50 can be contoured, when viewed in profile, so that first end portion 50, for example, does not lie in a common plane with shell 36. It is to be further understood that the present invention contemplates using any suitable technique for attaching first end portion 50 of seal 38 to shell 36. Such techniques may include permanently bonding seal 38 to shell 36, for example using adhesives or molding seal 38 onto shell 36, or attaching seal 38 to shell 36 using mechanical fasteners in a manner wherein seal 38 is selectively detachable from shell 36.

In the illustrated embodiment, a lock ring 52 slips over seal 38 and engages a lip defined on first end portion 50 of seal 38. Lock ring 52 attaches to shell 36 in any suitable manner. For example, the present invention contemplates providing locking tabs 56 on lock ring 52 that selectively attach to engaging portions of shell 36. When coupled to shell 36, seal 38 defines a chamber for receiving the nose of the user when respiratory mask 30 is donned by the user so that the user's airway is in fluid communication with the chamber.

Seal 38 also includes a second end portion 60 for sealing engagement with the face of a user, and a sidewall 62 extending between first end portion 50 and second end portion 60. According to the principles of the present invention, at least a portion of the exposed surface of seal 38 at second end portion 60 includes a contacting portion 64 structured to contact the skin of the user, and the contacting portion 64 comprises a component, which is effective to stimulate keratinocytes in the skin of the user. In certain embodiments, contacting portion 64 covers the entire exposed surface of seal 38. The component may be in the form of a composition which is overall deposited, applied or adhered to the surface of seal 38 to form contacting portion 64 which covers the entire exposed surface of seal 38. Alternately, the composition containing the component may be only partially deposited, applied or adhered to the surface of seal 38 to form contacting portion 64, which covers only sections or parts of the exposed surface of seal 38.

The partial coverage of the contacting portion 64 may result from the deposition or application process employed to form a component coating, whereby areas or portions of the exposed surface of the seal 38 are unintentionally not covered. Alternately, the partial coverage may be intentional such that the component is only deposited on or applied to or incorporated into selected, e.g., well-defined, areas of the seal 38, such that only these areas are covered by the contacting portion 64. For example, the seal 38 may be covered by the contacting portion 64 in selected areas where red mark formation is strongly present (e.g., the nose bridge region). As previously indicated, a respiratory mask may be used by a patient for a prolonged time and thus, red mark formations, skin damage, ulcers and pressure-sores can occur due to the prolonged pressure and shear loading of the skin by the mask cushion in combination with the build-up of moisture and heat in the skin due to the prolonged occlusion. In the illustrated, exemplary embodiment shown in FIGS. 1A, 1B, 2A and 2B, which, as described elsewhere herein, is a nasal mask, contacting portion 64 is structured to contact the area of the user generally around the nose including over the bridge of the nose.

Contacting portion 64 may be in the form of a layer, coating, thin film or the like. The component can be individually deposited or applied, or the component can be an ingredient in a coating composition and the coating composition can be deposited or applied to the surface of the seal 38 to form the contacting portion 64. Depending on the material of which the seal 38, or any other part(s) or portion(s), is composed, it is contemplated that the component may be at least partially absorbed into the seal 38 beneath its outer surface. The component and/or component-containing composition forms the coating that comprises the contacting portion 64, which contacts the skin of the user. The coverage and thickness of the coating can vary provided that the coverage and thickness is sufficient to essentially preclude contact of the (bare) surface of the seal 38 of the respiratory mask 30 with the skin of the user. The thickness may depend on the technique employed to deposit, apply or adhere the component-containing composition to the surface of the seal 38, or on the amount of the component that is desired to be in contact with the skin of the user.

A variety of known equipment and conventional coating techniques may be used to deposit, apply or adhere the component-containing composition to the surface of seal 38 to form contacting portion 64. Such techniques include, but are not limited to, spraying, brushing, wiping, rolling and the like. In certain embodiments, wherein the component individually does not readily and sufficiently adhere to the surface of the seal 38, it may be preferred to add the component as an ingredient of a coating composition, which contains an additive to improve adhesion. Alternatively, in these embodiments wherein the component individually does not readily and sufficiently adhere to the surface of the elastomeric material, it may be preferred to deposit or apply a base layer to the surface of the seal 38 and subsequently, deposit or apply the component to the surface of the base layer. The base layer material can be selected from a variety of materials that are known to enhance adherence of a coating to a surface of a substrate.

Further, it is contemplated that contacting portion 64 may be in the form of a component-containing film or layer that can be adhered to the surface of seal 38. An adhesive may be applied to at least one of the surface of contacting portion 64 and the surface of seal 38. In one embodiment, contacting portion 64 can be adhered to a substrate that is removable, e.g., a “peel n' stick” configuration.

As an alternative, or in addition, to depositing or applying the component-containing coating as contacting portion 64 to the surface of seal 38 of the respiratory mask 30, the component can be added to the material composition of seal 38 during its manufacture and the seal 38 (having the component therein), e.g., surface of seal 38 or second end portion 60, can contact the skin of the user. FIGS. 1B and 2B are identical to FIGS. 1A and 2A, with the following exception. In FIGS. 1A and 2A, the contacting portion 64 is shown as a coating deposited on or applied to the surface of seal 38 and in FIGS. 1B and 2B, the component is incorporated into the material composition of seal 38, e.g., second end portion 60, as shown by shading. FIGS. 1B and 2B are isometric and side elevational views, respectively, of a system 20 adapted to provide a regimen of respiratory therapy to a patient according to one embodiment of the present invention.

As seen in FIGS. 1B and 2B, system 20 includes a respiratory mask 30 (also referred to as a patient interface device) that includes seal 38 and a second end portion 60 for sealing engagement with the face of a user, and a sidewall 62 extending between first end portion 50 and second end portion 60. According to the principles of the present invention, at least a portion of the seal 38, e.g., second end portion 60, is structured to contact the skin of the user, and the seal 38 and/or second end portion 60 comprises a component, which is effective to stimulate keratinocytes in the skin of the user. The component is incorporated or embedded into the material composition of seal 38. There are a variety of conventional techniques that may be employed for incorporating or embedding the component into the material composition of seal 38. In one embodiment, the component is mixed or blended as an additive during preparation of the composition or formulation that makes up seal 38, prior to being molded into its end-use design. In another embodiment, the component is added, e.g., injected, into the material composition or into a mold during the molding or casting process.

In one embodiment, during preparation of the composition or formulation that makes up seal 38, the component is added and thus, when the material composition is subjected to a molding process the component is imparted into the molded product, e.g., seal 38, and the surface of the seal 38, e.g., second end portion 60, contacts the skin of the user. The component can be uniformly dispersed throughout the material composition of seal 38. For example, when the component is in the form of a liquid, the liquid can be mixed with the material composition of seal 38, such that the component is uniformly distributed therein. In certain embodiments, the seal 38 may have a greater concentration of component on or near the surface, e.g., second end portion 60, as compared to the remainder of seal 38. For example, when the component is in the form of particles, the particles can be distributed on or near the surface of the seal 38, e.g., second end portion 60.

As above-described, the component, which is effective to stimulate keratinocytes in the skin of the user, is in the form of contacting portion 64 positioned on the surface of seal 38 or incorporated into the material composition of seal 38, e.g., second end portion 60. It is contemplated, however, that the component can be combined with any part(s) or portion(s) of the mask that contacts the skin of the user or any part(s) or portion(s) of the mask that does not contact the skin of the user but is close to or near the external tissue of a user and is structured to release the component from said part or portion to contact the external tissue in an amount sufficient to stimulate keratinocytes in the skin of the user. In one embodiment, the component can be released into the atmosphere. In another embodiment, the component can be released into an air flow path, such as, but not limited to, air that flows from an air source connected to a respiratory mask. For example, the component may combined with a respiratory therapy device by connecting a component-containing reservoir with the device, such that the reservoir provides a release of the component into the therapy airflow path and/or the ambient air. The component can be released intermittently or continuously. In certain embodiments, a control mechanism can be connected to the reservoir to provide for a timed release of the component.

The component for use in the invention can be selected from those materials that are known to activate cellular receptors, e.g., olfactory receptors, expressed in skin cells, e.g., keratinocytes, which results in accelerated wound healing to repair skin damage and preclude or heal ulceration of the skin. A non-limiting example of a suitable component is sandalwood, which has been shown as an effective agonist of OR2AT4 (i.e., one of the olfactory receptors found in keratinocytes) resulting in proliferation and migration of keratinocytes, and acceleration of re-epithelialization. Sandalwood is known and commercially available under the trade names Sandalore® and Brahmanol®. The amount and/or concentration of component can vary, and should be such that the component, which is in contact with the skin, is effective stimulate keratinocytes in external tissue and to activate the wound healing mechanism. In certain embodiments, the concentration of the component is from about 1 μM to greater than about 1 mM.

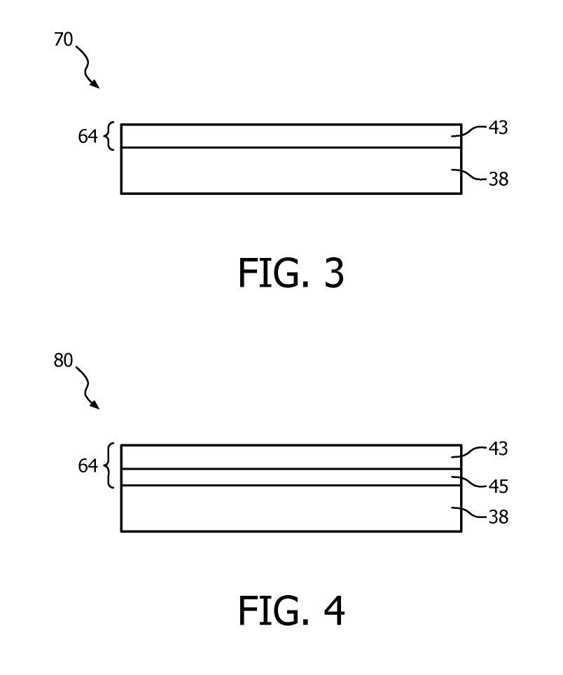

FIG. 3 is a schematic showing a cross-section view of a component-coated part 70, which includes seal 38 (as shown in FIGS. 1A and 2A) having a component-containing coating 43 applied to the surface of seal 38. The component-containing coating 43 serves as contacting portion 64 (as shown in FIGS. 1A and 2A). As mentioned above, it is contemplated that the component-containing coating 43 can be composed of the component individually or the component-containing coating 43 can be composed of a coating composition wherein the component is an ingredient of the coating composition. FIG. 4 is a schematic showing a cross-section view of a component-coated part 80, which includes seal 38 having a base coating 45 applied to the surface of seal 38 and the component-containing coating 43, shown in FIG. 3, applied to the surface of the base coating 45. In FIG. 4, the combination of the base coating 45 and the component-containing coating 43 serves as contacting portion 64. As previously described herein, forming a component coating 43 on the exterior surface of the seal 38 is advantageous as it serves as an agonists of olfactory receptors, e.g., OR2AT4, to activate a cellular signaling cascade that triggers the proliferation and migration of keratinocytes which accelerates re-epithelialization, to enhance healing of wounds that may result from prolonged wear of a respiratory therapy mask by a user.

It may be desired to control or slow the release of the component such that the component can be substantially continuously released from the contacting portion 64 or the surface of seal 38, e.g., second end portion 60, for the mission life of the contacting portion 64 or the seal 38 of the respiratory mask 30. In one embodiment, the component can be combined with one or more additives that are known in the art for this purpose. In other embodiments, the component can be modified, such as, by treating or encapsulating the component with a material that provides at least partial retention of the component and is capable of degrading over time to effectuate a controlled or slow release of the component from the material. Suitable treating and encapsulation materials, such as polymers, can be selected from those known in the art.

As noted elsewhere herein, seal 38 is, in the exemplary embodiment, made of a soft, flexible, cushiony, elastomeric material. In one exemplary, non limiting embodiment, the specific material may be silicone having a durometer of 40 Shore A. In another exemplary, non limiting embodiment, the specific material may be liquid silicone rubber having a durometer of 5 Shore A.

While the exemplary embodiments described above employ combining the component with seal 38 and/or the contacting surface 64, it is to be understood that these are not meant to be limiting. For example, the contacting surface 64 comprising the component as described herein may form part of an in-lay or patch (e.g., disposable) that is coupled to the main body of seal 38 to form seal 38. It is to be further understood that the component as described herein is not limited to use on or in seal 38, but may be combined with, i.e. applied to or incorporated in, other skin contacting surfaces/components of a patient interface device. For example, a component as described herein may be employed on or in forehead pad 46 or other skin contacting components that may be employed with respiratory mask 30, such as cheek pads/supports or chin pads/supports.

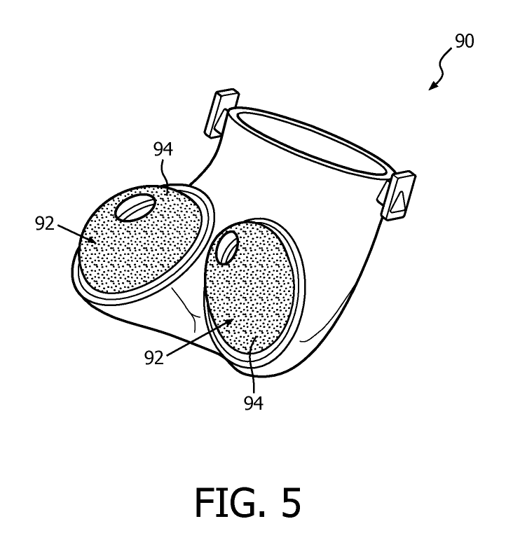

In addition, the component as described herein may be used on or in contacting portions of different types of patient interface devices, such as, without limitation, the nasal cushion 90 shown in FIG. 5, which includes a pair of laterally spaced dome-shaped nares elements 92. As seen in FIG. 5, each dome-shaped nares elements 92 has a contacting portion 94 having the component as described herein provided over substantially the entire surface area thereof. It is to be understood that the contacting portions 94 can, alternatively, be provided over only a portion of each nares element 92 in a select pattern or select locations.

It is also to be understood that the component as described herein is not limited to use on or in patient interface devices. Instead, the component can be used in combination with external skin contacting portions on a wide variety of devices. Non-limiting examples include, earpieces, headsets, googles, e.g., swim googles and ski googles, wristwatches, wristbands, jewelry, helmets, baby bottles, pacifiers, diapers, breast pumps, bedding, e.g., sheets, pillows, mattress covers and the like, seats, e.g., bicycle seats, wheel chair seats and the like, footwear, e.g., sandals, diving flippers, and the like, as well as the handles of a wide variety of articles, such as, but not limited knives, shovels, lawn mowers, snow blowers, vehicle steering wheels, suitcases, briefcases, purses, pet leashes, strollers, baby carriers, bicycles, joysticks, sports equipment, e.g., baseball bats, tennis racquets, lacrosse sticks, exercise equipment, e.g., weight machines and barbells, and the like.

The present invention provides numerous advantageous over known respiratory therapy devices and related devices that can cause skin wounds or damage from contact with the user's skin. For example, in the present invention the component can be applied to the skin through use of the device and no additional action is required by the user to apply the component. That is, often a separate composition needs to be applied to the skin and, in particular, to portions of the skin that are in contact with a device. Further, the component is applied to skin that may not be damaged or wounded and as a result, the component may be effective to reduce or prevent the formation of damage or wounds caused by contact of the skin with a device. Furthermore, the components employed in the invention may inherently provide an odor that is pleasing to the user as compared to conventional pharmaceutical skin compositions that do not have a pleasing odor associated therewith.

It can be appreciated that the present invention provides for the use of a component, such as sandalwood, in a device, such as a respiratory mask, that is structured to contact the skin or other external tissue of the user for prolonged periods that provides keratinocyte stimulation and/or improved wound prevention or wound healing for the user.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A respiratory mask structured to directly contact external tissue of a user during use thereof, comprising:
a wound preventive or healing portion that comprises a sandalwood component effective to stimulate keratinocytes in the external tissue, the portion in a form selected from the group consisting of:
a layer, coating or thin film at least partially deposited on, applied to or adhered to an exposed surface of one or more parts of said mask;
an additive to a coating composition at least partially deposited on, applied to or adhere to an exposed surface of one or more parts of said mask to form a layer, coating or thin film; and
an ingredient in a material composition that forms one or more parts of said mask.

2. A respiratory mask according to claim 1, wherein the portion is a contacting portion that is structured to directly engage the external tissue of the user.

3. A respiratory mask according to claim 1, wherein the portion is a non-contacting portion that is structured to release the sandalwood component from the portion and to indirectly engage the external tissue of the user.

4.-7. (canceled)

8. A respiratory mask according to claim 2, wherein the sandalwood component is present in the contacting portion in an amount from 1 μM to greater than 1 mM.

9. A method of making a respiratory mask structured to directly contact external tissue of a user during use thereof, comprising:
obtaining a sandalwood component effective to stimulate keratinocytes in the external tissue; and
providing a wound preventive or healing portion for the mask comprising the sandalwood component, the portion being in a form selected from the group consisting of:
a layer, coating or thin film at least partially deposited on, applied to or adhered to an exposed surface of one or more parts of said mask;
an additive to a coating composition at least partially deposited on, applied to or adhere to an exposed surface of one or more parts of said mask to form a layer, coating or thin film; and
an ingredient in a material composition that forms one or more parts of said mask.

10.-11. (canceled)

12. The method according to claim 9, wherein the portion is structured to at least partially release the sandalwood component to contact the external tissue.

13. (canceled)

14. A method for preventing or healing wounds resulting from direct contact of external tissue of a user by a respiratory mask during use thereof, comprising:
providing the respiratory mask;
obtaining a sandalwood component effective to stimulate keratinocytes in the external tissue; and
providing a wound preventive or healing portion for the mask comprising the sandalwood component, the portion being in a form selected from the group consisting of:
a layer, coating or thin film at least partially deposited on, applied to or adhered to an exposed surface of one or more parts of said mask;
an additive to a coating composition at least partially deposited on, applied to or adhered to an exposed surface of one or more parts of said mask to form a layer, coating or thin film; and
an ingredient in a material composition that forms one or more parts of said mask.
